(19) United States
(12) Patent Application Publication
(10) Pub. No.: US 2002/0183812 A1
(43) Pub. Date: Dec. 5, 2002

(54) Multi-functional therapy station

(76) Inventor: Huguette Long, Ottawa (CA)

Correspondence Address: Marks & Clerk, Station B, P.O. Box 957, Ottawa, ON, K1P 5S7, CA

(21) Appl. No.: 10/157,225

(22) Filed: May 30, 2002

Related U.S. Application Data
(60) Provisional application No. 60/333,480, filed on Nov. 28, 2001.

(30) Foreign Application Priority Data
May 30, 2001 (CA) 2,349,075

(51) Int. Cl. A61F 7/00

(52) U.S. Cl. 607/96

(57) ABSTRACT

A multi-functional therapy station for use in dry, wet and steam therapy is described. The multi-functional station includes a table with shallow side walls. Guiding tracks are located on the side walls and a plurality of flexible panels are slidably located in the guiding track to form a semi-rigid dome. The table includes means to introduce steam into the dome when used as a steam therapy station. The flexible panels can be removed and the table used for dry and/or wet therapy.

This invention claims the benefit of U.S. Provisional No. 60/333,480 filed Nov. 28, 2001.

### FIELD OF THE INVENTION

This invention relates to a therapy station and more particularly to a multi-functional therapy table which can be used in wet, dry and steam therapy sessions.

### BACKGROUND OF THE INVENTION

It is well known that message therapy treatments provided in dry, as well as wet and steam environments are beneficial to one's health. Such therapy is typically administered by physical therapists, massage therapists, chiropractors, nurses, acupuncturists, etc in clinics or massage establishments. Traditionally, dry therapy treatments are provided on massage tables that are commonly located in a dry room. Practitioners of the alternative health industry are very familiar with the restrictions of a message table when they are faced with a situation in which a wet treatment or alternatively a steam treatment would augment the dry therapy. Usually, a dry treatment is provided in one room whereas a hydro table or a wet table for wet therapy is located in a wet room facility and requires plumbing for drainage purposes. Alternatively the wet table may be in a room with a sit down steam cabinet located in a shared room. Accordingly, practitioners and spa establishments alike are often restricted to employing popular treatments such as massage, hydro therapy or steam treatments on different tables and/or in separate locations of their establishment. This requires considerable outlay for expensive equipment, not to mention the inefficient procedures necessitated by moving the patient or client from one facility to the other.

Examples of such therapeutic bathing apparatus may be found in U.S. Pat. No. 5,441,529 which issued Aug. 15, 1995 to Dorsch; U.S. Pat. No. 5,645,578 which issued Jul. 8, 1997 to Daffer et al. and U.S. Pat. No. 6,057,005 which issued Dec. 5, 2000 to Lu.

There have been attempts to combine at least wet and steam tables and these are described in, for example, U.S. Pat. No. 3,885,557 which issued May 27, 1975 to Varea and U.S. Pat. No. 5,425,753 which issued Jun. 20, 1995 to Wege.

The U.S Pat. No. 5,425,753 discloses a table which uses a portable steam unit and a cloth canopy located over the table. The cloth canopy has a number of supporting or reinforcing poles which maintain the canopy in a formed structure over the table and which may be removed using suitably located web loops on the canopy.

The difficulty with this device is that the cloth canopy is difficult to store particularly when it is wet from a session and, also, difficult to dry between sessions.

Accordingly, there is a need for a multi-functional therapy station in which a massage therapist has convenient access to a patient when functioning in the dry state and which can easily be converted to a wet or steam table when alternative treatment would benefit the patient.

Therefore, in accordance with a first aspect of the present invention there is provided a multi-functional therapy station comprising: a table having supporting legs and shallow side walls; means in the table for use in introducing steam and for draining liquids therefrom; a removable table top resting on ribs on the table; a guiding track on opposed lateral side walls; a plurality of flexible panels extending over the table top, with opposed side edges slidably engaging the guiding track to form a segmented dome; and a foot shield for closing one end of the dome.

### BRIEF DESCRIPTION OF THE DRAWINGS

The invention will now be described in greater detail with reference to the attached drawings wherein;

FIG. 1 is a perspective view of the therapy station;

FIG. 2 is a perspective view of the station without the flexible panels.

FIG. 3 illustrates the table top in a tilted orientation;

FIG. 4 illustrates the ribs in the table;

FIG. 5 shows the means for supporting the table top in a tilted position;

FIG. 6 shows the foot shield; and

FIG. 7 illustrates the guiding tracks removed from the side walls.

### DETAILED DESCRIPTION OF THE INVENTION

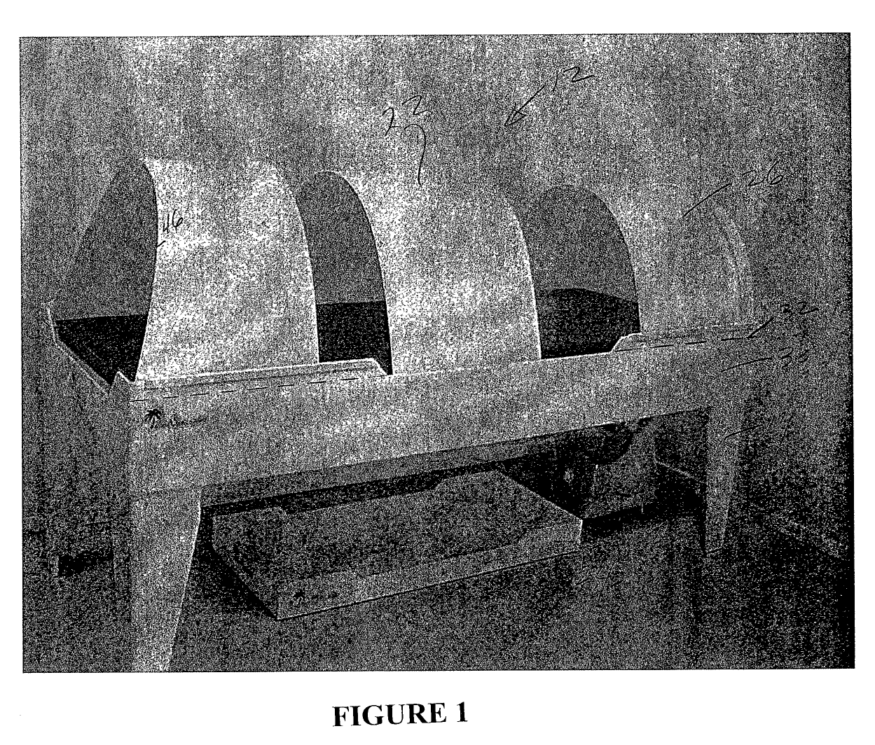

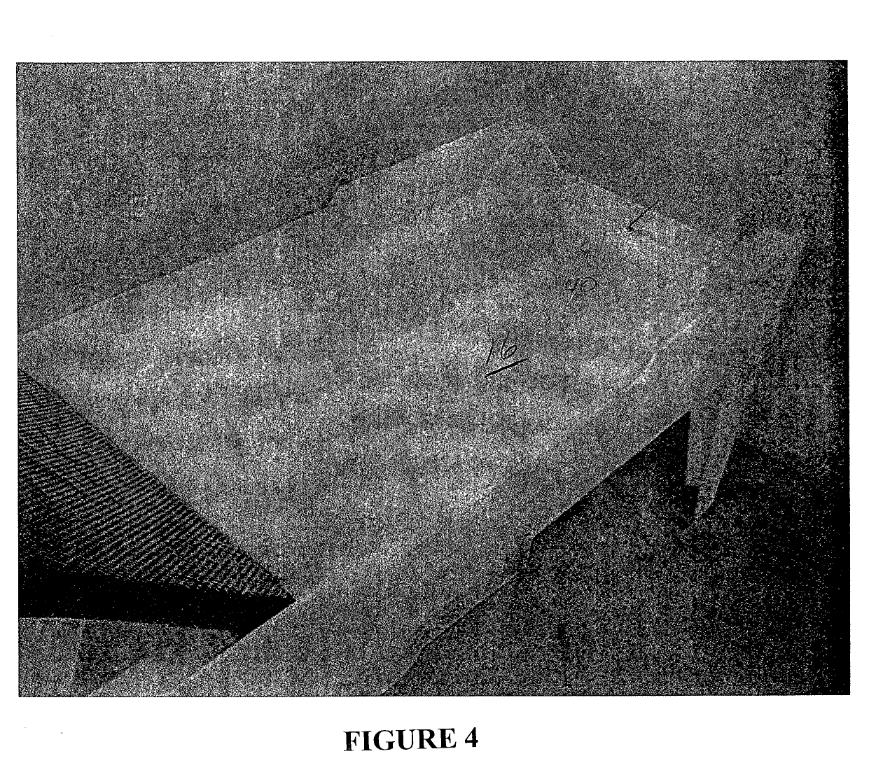

As shown in FIG. 1 the therapy station 12 includes supporting legs 14 and a table top which rests on the upper surface 16 of the table as best seen in FIG. 4. The table top 18, is upholstered and covered and is removably located on the surface of the table base. The upholstered covering can also be covered with a water proof sheet placed over it when the station is to be used for wet or steam applications.

Each leg 14 is provided with separately adjustable height adjusting means 20 to accommodate an uneven floor or to tilt or slightly raise one end of the table relative to the other. Height adjusting members for table legs and the like are well known and further description thereof is believed unnecessary.

Also shown in FIG. 1 are flexible panels 22 which are slidable lengthwise in a manner which will be described later. Also shown in FIG. 1 is a trolley or storage compartment 24 which is located under table and which is designed to store the flexible panels 22 when they are not in use. Preferably the trolley will have swivel wheels to allow the trolley to be moved from under the table for easy access to the panels. The trolley will also be used to store the foot shield 26 when not in use.

Also shown in FIG. 1 is a compact steam generator 28 which is used to generate steam for use in a steam therapy treatment.

As shown in FIG. 1, side walls 30, have a middle section with reduced height but it is to be understood that in a preferred embodiment the side walls are at the same reduced height throughout their length as shown by dotted line 32.

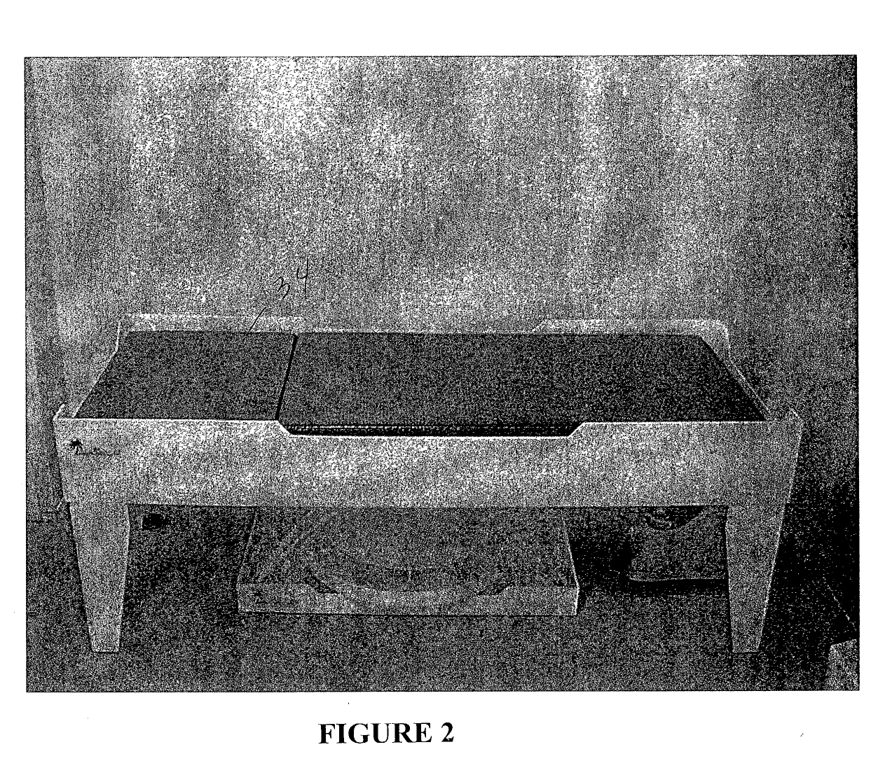

FIG. 2 illustrates the therapy station with flexible panels 22 and foot shield 26 removed.

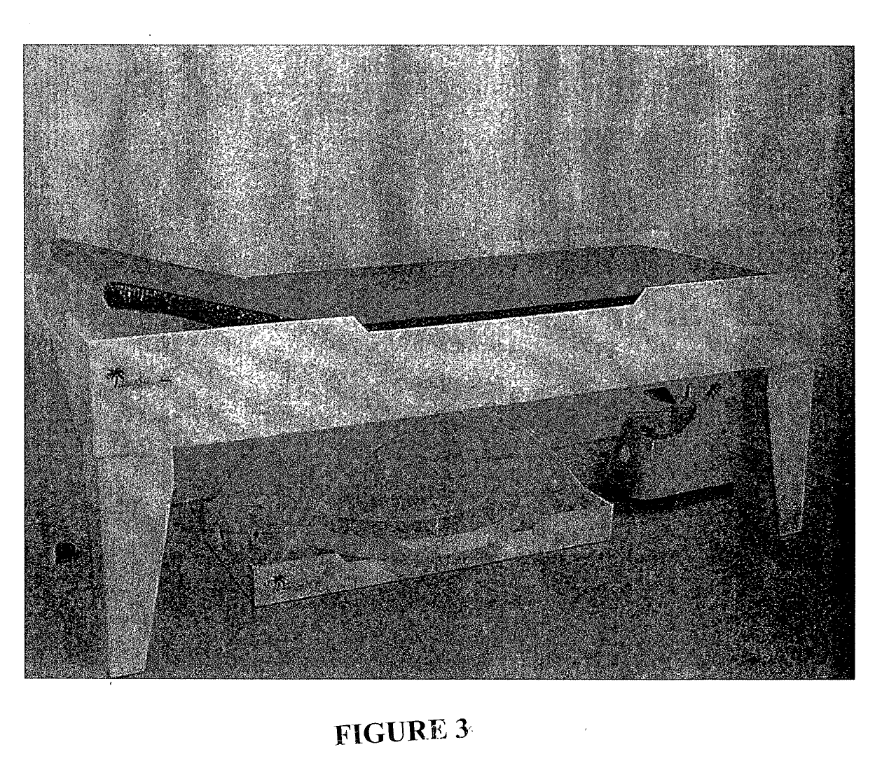

FIG. 2 also shows that the table top 18 has a hinged portion 34 which allows one end, typically the head end, to be tilted at varying angles as shown more clearly in FIG. 3.

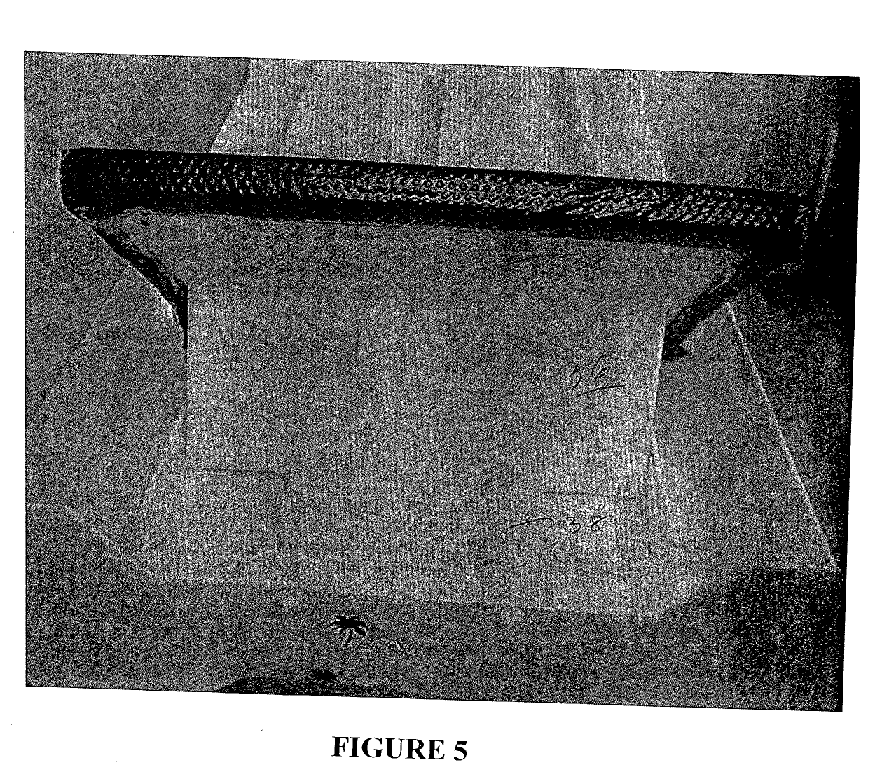

FIG. 5 illustrates an end view of the table with the table top 18 in a tilted position and retained in that location by means of the tilting board 36 which is positioned in slots 38 on both the underside of the table top and on the top of the table itself.

As shown more clearly in FIG. 4 the top surface 16 of the table itself is provided with a plurality of supporting ribs 40 which serve to retain the table top somewhat elevated from the actual table surface. As shown in FIG. 5 the table top is slightly narrower than the table base itself thereby providing channels 42 along the outer edge of the table top and the shallow side walls. This channel serves to store liquids which may be used in a wet treatment session, the channels serving to direct the fluid to one end of the table. This flow is enhanced by the fact that when in the wet mode the head end of the table may be slightly elevated relative to the foot end so that the liquid will all flow to the front end. A drain 44 which may be seen in FIG. 4 permits the excess liquid to be drained from the table and connected to a drainage system or collected in a suitable container for disposal.

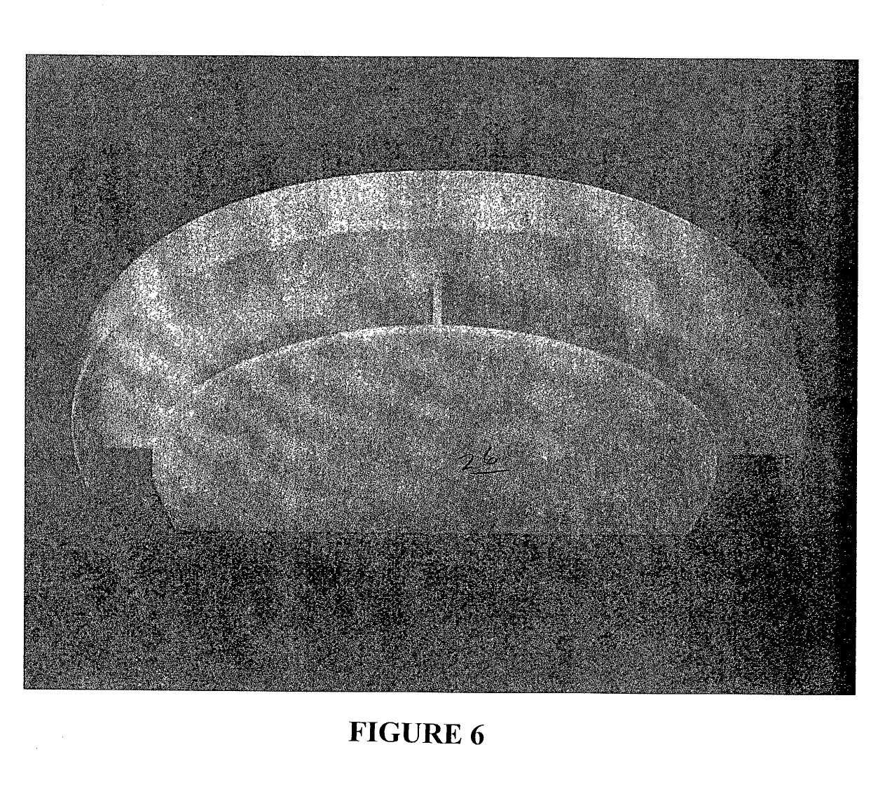

FIG. 6 illustrates the foot shield 26 which may also be seen in FIG. 1 and which closes the foot end of the table and, of course, provides extra rigidity to the dome. Although the flexible panels 22 are shown as being separated in FIG. 1 it is to be understood that they do, in fact, overlap in operation.

A steam veil (not shown) can be draped over the dome, formed by the panels 22 and, if desired, placed close to the patients neck. As shown in FIG. 1 the front edge 46 of the front panel has a sloping contour which leads to better positioning of the veil.

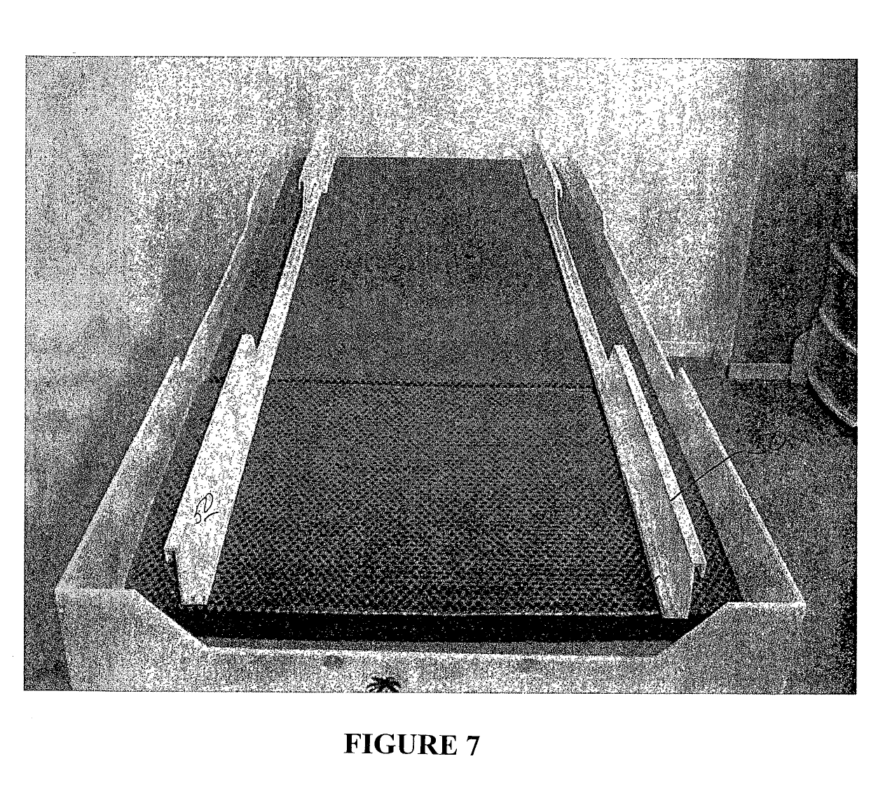

FIG. 7 illustrates the guiding tracks 50 in a location removed from the side walls 30. The guiding tracks 50 are designed to hook securely onto the lateral frames or side walls 30 of the table and are dimensioned such that when in position occupy the lateral channel. These guiding tracks 50 provides support and resistance for the flexible panels 22 to be systematically positioned into their corresponding grooves 52 of the guided track. When the panels are strategically placed into position along the guiding tracks a segmented dome is bridged above the resting table top as shown in FIG. 1. The foot shield 26 (FIG. 6) comprises a posterior steam shield and a projected anterior steam shield and is lowered on to the base of the table to contact the flexed panel positioned at the foot of the table.

It will be apparent to one skilled in the art that a suitable fixture will be installed at the foot of the table and will protrude through the base for use in connecting the steam generator and will function as the initial point of steam distribution. The drain hole 44 is, of course optional and will only be required when the table is used for wet treatments.

The table can be constructed of high density polyethylene and plastic with appropriate welding at joining seems in order to render the table water proof. The flexible panels and foot shields can be constructed of flexible polyethylene as is known in the plastics art.

It is also to be understood that the table top as shown can be replaced with other types of table tops should such be required for certain message procedures.

While certain embodiments of the invention have been described and illustrated it would be apparent to one skilled in the art that numerous changes can be made without departing from the basic concept of the invention. It is to be understood, however, that such changes will fall within the true scope of the invention as defined by the appended claims.

1. A multi-functional therapy station comprising: a table having supporting legs and shallow side walls; means in the table for use in introducing steam and for draining liquids therefrom; a removable table top resting on ribs on the table; a guiding track on opposed lateral side walls; and a plurality of flexible panels extending over the table top, with opposed side edges slidably engaging the guiding track to form a segmented dome; and a foot shield for closing one end of the dome.

2. A multi-functional therapy station as defined in claim 1 further having a removable steam veil extending over the dome.

3. The multi-functional therapy station as defined in claim 1 wherein said table top has a hinged portion which may be tilted.

4. The multi-functional therapy station as defined in claim 1 wherein the table top has an upholstered surface.

5. The multi-functional therapy station as defined in claim 4 wherein said upholstered surface has a removable water proof cover.

6. The multi-functional therapy station as defined in claim 1 wherein the supporting legs have height adjustment means.

7. The multi-functional therapy station as defined in claim 1 further having a wheeled trolley for storing said flexible panels and said foot shield when not in use.

8. The multi-functional therapy station as defined in claim 7 wherein said flexible panels partially overlap in an operational mode.

9. The multi-functional therapy table as defined in claim 8 wherein said foot shield has a posterior steam shield and an inwardly projecting interior steam shield, one of the flexible panels engaging said steam shields to close the end of the dome.
